(19) United States
(12) Patent Application Publication
(10) Pub. No.: US 2025/0049337 A1
(43) Pub. Date: Feb. 13, 2025

(54) METHOD AND SYSTEM FOR ANALYZING PERFUSION PARAMETERS OF SKIN

(71) Applicant: KONINKLIJKE PHILIPS N.V., EINDHOVEN (NL)

(72) Inventors: Tobias Wissel, Lübeck (DE); Sven Kroenke-Hille, Hamburg (DE)

(21) Appl. No.: 18/718,472

(22) PCT Filed: Dec. 17, 2022
(86) PCT No.: PCT/EP2022/086518

§ 371 Date: Jun. 11, 2024

Related U.S. Application Data
(60) Provisional application No. 63/291,984, filed on Dec. 21, 2021.

(51) Int. Cl. A61B 5/026; A61B 5/00

(52) U.S. Cl. CPC A61B 5/0261; A61B 5/441

(57) ABSTRACT

A system and method are provided for analyzing perfusion parameters of skin. The method includes acquiring a multi-band reflectance image of skin; delineating at least one clinically relevant spatial component of the multi-band reflectance image of the surface, where the at least one clinically relevant spatial component has substantially homogenous optical properties; performing reconstruction on the at least one clinically relevant spatial component using a corresponding at least one tailored reconstruction algorithm, respectively, where the at least one tailored reconstruction algorithm is specific to the at least one clinically relevant spatial component; and outputting estimated perfusion parameters of the skin for the at least one reconstructed clinically relevant spatial component from the at least one tailored reconstruction algorithm.

### BACKGROUND

Skin perfusion parameters, such as blood volume, flow and oxygenation, are analyzed in a spatially resolved manner for assessing severity of peripheral vascular diseases and for monitoring the impact of interventions on perfusion and wound healing processes. Optical contactless methods, such as multispectral and hyperspectral imaging, offer advantages for the clinical routine as they are easy to use, fast and avoid infections due to remote surface sensing. However, the optical contactless methods provide only indirect measurements of the perfusion properties of interest, and therefore rely on reconstruction/inference algorithms.

The reconstruction/inference algorithms include translating backscattered spectra to clinically meaningful perfusion parameters, where the backscattered spectra may consist of diffuse reflectance for a set of wavelengths. Translating the backscattered spectra to the clinically meaningful perfusion parameters relies on physically modelling optical properties of the skin. The modeling may be realized by learning from a limited amount of real-world data or from simulated data, in which Monte-Carlo models of varying skin and/or perfusion properties yield corresponding reflection spectra. In both cases, a forward model, such as artificial neural network performing a regression task, is trained to reconstruct desired perfusion parameters from the spectra.

Accordingly, the optical contactless methods are only able to account for spatial skin heterogeneities up to a certain degree due to the strong variability of possible input spectra. These input spectra exhibit the different reflectance and/or absorbance behavior for various regions, including healthy skin, wounds and surface veins, as well as disturbances such as hairs, moles, and pimples, for example. The variability may result in ambiguities in the input spectra, which cause sub-optimal prediction accuracy of corresponding perfusion parameters. Even where there are no ambiguous input spectra, modelling all the expected input variability has limitations due to limited numbers of samples from the theoretical input distribution in real-world data scenarios or due to limitations in the physical complexity of Monte-Carlo skin models, for example. The Monte-Carlo skin models, in particular, may include spatial structures that can be recognized as anomalies in a spectral surface map, but there is no value or reasonable effort-value trade-off in modelling them. Such anomalies may include telangiectasia, follicles, moles, and age spots, for example.

### SUMMARY

According to a representative embodiment, a method is provided for analyzing perfusion parameters of skin. The method includes acquiring a multi-band reflectance image of skin of a subject; delineating at least one clinically relevant spatial component of the multi-band reflectance image of the skin, where the at least one clinically relevant spatial component has substantially homogenous optical properties; performing reconstruction on the at least one clinically relevant spatial component using a corresponding at least one tailored reconstruction algorithm, respectively, where the at least one tailored reconstruction algorithm is specific to the at least one clinically relevant spatial component; and outputting an estimated perfusion parameters of the skin for the at least one reconstructed clinically relevant spatial component from the at least one tailored reconstruction algorithm.

According to another representative embodiment, system for analyzing perfusion parameters of skin. The system includes an imaging system configured to acquire a multi-band reflectance image of skin; at least one processor coupled to the imaging system to receive the multi-band reflectance image of the skin of a subject; at least one non-transitory memory storing instructions; and a display. When executed by the at least one processor, the instructions cause the at least one processor to: delineate at least one clinically relevant spatial component of the multi-band reflectance image of the skin, where the at least one clinically relevant spatial component has substantially homogenous optical properties; perform reconstruction on the at least one clinically relevant spatial component using a corresponding at least one tailored reconstruction algorithm, respectively, where the at least one tailored reconstruction algorithm is specific to the at least one clinically relevant spatial component; and output estimated perfusion parameters of the skin for the at least one reconstructed clinically relevant spatial component from the at least one tailored reconstruction algorithm. The display is configured to display the estimated perfusion parameters for the at least one reconstructed clinically relevant spatial component in a spatially resolved manner.

According to another representative embodiment, non-transitory computer readable medium is provided that stores instructions for analyzing perfusion parameters of skin of a subject. When executed by at least one processor, the instructions cause the at least one processor to receive a multi-band reflectance image of the skin; delineate at least one clinically relevant spatial component of the multi-band reflectance image of the skin, where the at least one clinically relevant spatial component has substantially homogenous optical properties; perform reconstruction on the at least one clinically relevant spatial component using a corresponding at least one tailored reconstruction algorithm, respectively, where the at least one tailored reconstruction algorithm is specific to the at least one clinically relevant spatial component; output estimated perfusion parameters of the skin for the at least one reconstructed clinically relevant spatial component from the at least one tailored reconstruction algorithm; and cause a display of the estimated perfusion parameters for the at least one reconstructed clinically relevant spatial component in a spatially resolved manner.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system for analyzing perfusion parameters of skin, according to a representative embodiment.

FIG. 2 shows illustrative images of a human foot subjected to delineation of homogenous regions, according to a representative embodiment.

FIG. 3 shows illustrative images of a delineated homogeneous region of an estimated perfusion parameter map, according to a representative embodiment.

FIG. 4 shows illustrative images of spatially irrelevant spatial components subjected to inpainting, according to a representative embodiment.

FIG. 5 is a flow diagram showing a method of analyzing perfusion parameters of skin, according to a representative embodiment.

FIG. 6 is a flow diagram of a method of inpainting the at least one clinically relevant spatial component for analyzing perfusion parameters of skin, according to a representative embodiment.

FIG. 7 is a flow diagram of a method of monitoring estimated perfusion parameters that are spatially accumulated over time for analyzing perfusion parameters of skin, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As mentioned above, measuring perfusion parameters, such as blood volume, blood flow and oxygenation, using multispectral or hyperspectral imaging (collectively referred to as “multi-band imaging”) is problematic. First, the perfusion parameters are measured indirectly, and therefore need to be reconstructed or mapped from the measured spectra. Such mapping of spectra to perfusion parameters is typically based on a reconstruction algorithm (e.g. artificial neural network) trained on spectrum/perfusion parameter pairs, which may be referred to as “data samples.” The data samples need to sufficiently cover all relevant situations occurring in actual skin imaging. However, due to the different reflectance and/or absorbance of different structures, including healthy skin tissue, wounds, surface veins, hairs, moles and pimples, for example, there is significant variability in the input data and, for a given patient, some of these structures may occur in the same image. Also, not all of the structures may be relevant for the perfusion analysis as such. In practice, an exhaustive training data set is difficult to obtain by alternative measurement methods or Monte-Carlo simulations for different tissue compositions.

Second, even when the variability is covered by the training data, it is still difficult to train an overall accurate model that performs well on all of the different structures. Moreover, fundamental problems in the reconstruction may occur. For example, different spatial structures and tissue compositions may result in nearly indistinguishable spectra at the selected wavelengths. In this case any reconstruction algorithm operating on the bare spectra would give the same perfusion parameter prediction for the different structures. Third, in addition to degraded accuracy, some structures such as hairs, moles and pimples may lead to a localized disturbance of the estimated perfusion parameter map, leading to a suboptimal visualization of the perfusion pattern. In summary, surface heterogeneities in the spatial surface structure give rise to two different scenarios: (i) need for tailored processing due to relevance of a structure, or (ii) need for properly handling disturbances that are clinically irrelevant for the desired perfusion measurements.

Generally, the various embodiments described herein provide a system and method for measuring perfusion parameters, such as blood volume, flow and oxygenation. An optical contactless imaging system is used to acquire a multi-band reflectance image (spectral image) of skin or other tissue surface (collectively referred to as “skin surface”). The optical contactless imaging system includes a broadband light source and a spatially resolved camera system able to resolve a certain number of appropriately selected wavelengths in the multi-band reflectance image. Relevant spatial structures may be marked in the acquired image, or in a spatially aligned RGB-value and/or grey-value image, using a user interface or a marking algorithm. At least one reconstruction algorithm may be applied to at least one clinically relevant spatial structure of the marked spatial structures to provide estimated perfusion parameters from input spectra (obtained at a single pixel or a set of neighboring pixels). The estimated perfusion parameters are more accurate and/or clinically meaningful as compared to simply using, e.g., a single reconstruction algorithm, as in conventional techniques.

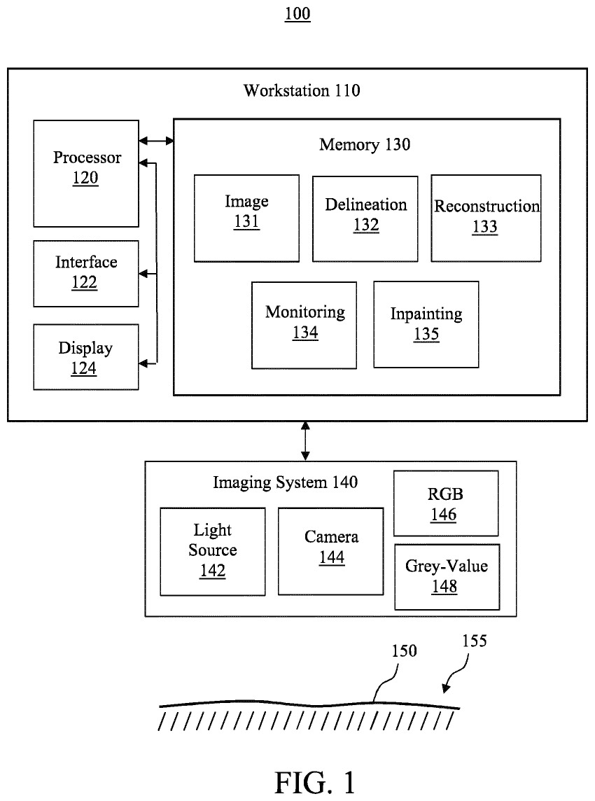

FIG. 1 is a simplified block diagram of a system for analyzing perfusion parameters of skin, according to a representative embodiment.

Referring to FIG. 1, system 100 includes a workstation 110 for implementing and/or managing the processes described herein. The workstation 110 includes one or more processors indicated by processor 120, a user interface 122, display 124 and one or more memories indicated by memory 130. The processor 120 interfaces with an imaging system 140 through an imaging interface (not shown), which provides optical data from skin 150 of a subject 155.

In the depicted embodiment, the imaging system 140 is an optical contactless imaging system that includes a broadband light source 142 and a spatially resolved camera 144 for performing multi-band imaging of the skin 150 to obtain a multi-band reflectance image. The broadband light source 142 provides at least two frequency bands measured in the reflectance imaging. The multi-band reflectance image includes at least two wave-lengths in an optical, near infrared and/or near ultra-violet range, and may be a multi-spectral image or a hyperspectral image, for example. Optionally, the imaging system 140 may also include an RGB-value camera 146 and/or a grey-value camera 148, in addition to or as part of the spatially resolved camera 144, for acquiring additional RGB-value images and/or grey-value images of the skin 150, respectively. The RGB-value camera 146 and/or the grey-value camera 148 are spatially aligned with the spatially resolved camera 144. Alternatively, RGB-value images and/or grey-value images may be derived from the images of the skin 150 provided by the spatially resolved camera 144. The imaging system 140 may include optical filters, such as polarization or wavelength filters (not shown).

As discussed below, local spectra output by the imaging system 140 are mapped by the processor 120 to hemodynamic parameters (e.g. blood volume, flow and oxygenation), e.g., output by reconstruction module 133 taking spatial factors into account, which can lead to more accurate hemodynamic parameter estimations as compared to reconstruction algorithms that do not take the spatial factors into account. Examples of such spatial factors include tissue type (e.g., wound tissue vs. healthy skin tissue) and the presence and/or absence of moles. In alternative embodiments, the imaging system 140 may provide another type of imaging, such as spectral CT, for example, to provide indirect measurements of physiological parameters by multi-band imaging, when the mapping from the multi-band image to parameters is unknown.

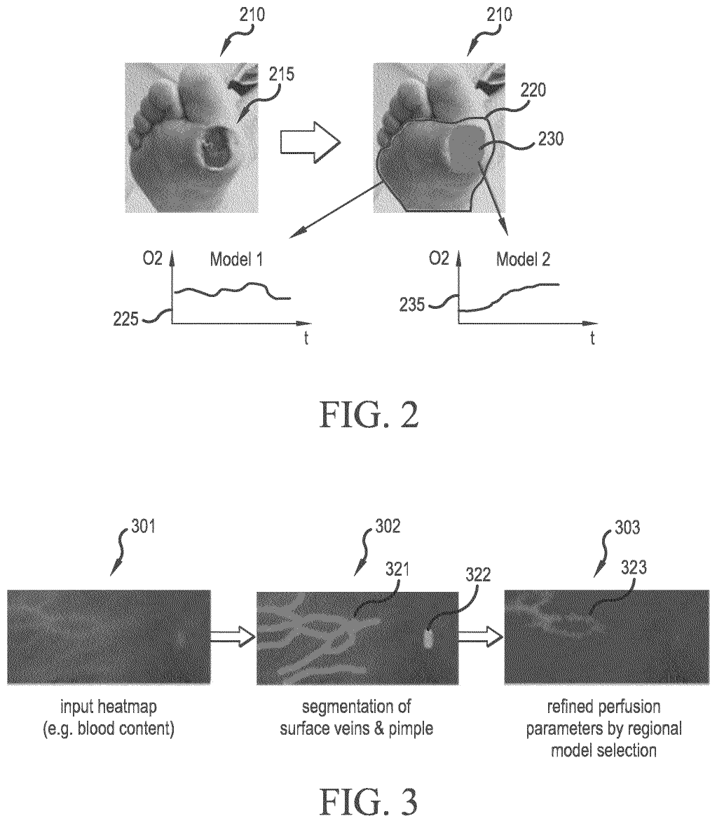

The memory 130 stores instructions executable by the processor 120. When executed, the instructions cause the processor 120 to implement one or more processes for analyzing perfusion parameters of skin, described below with reference to FIG. 2, for example. For purposes of illustration, the memory 130 is shown to include software modules, each of which includes the instructions corresponding to an associated capability of the system 100. It is understood, though, the depiction is non-limiting such that instructions for performing the various functions may be arranged or group in any manner within the memory 130 and/or additional memories, without departing from the scope of the present teachings.

The processor 120 is representative of one or more processing devices, and may be implemented by field programmable gate arrays (FPGAs), application specific integrated circuits (ASICs), a digital signal processor (DSP), a general purpose computer, a central processing unit, a computer processor, a microprocessor, a microcontroller, a state machine, programmable logic device, or combinations thereof, using any combination of hardware, software, firmware, hard-wired logic circuits, or combinations thereof. The processor 120 may be implemented as a single processor, multiple processors, or parallel processors. Multiple processors may be included in, or coupled to, a single device or multiple devices. The term “processor” as used herein encompasses an electronic component able to execute a program or machine executable instruction. A processor may also refer to a collection of processors within a single computer system or distributed among multiple computer systems, such as in a cloud-based or other multi-site application. Programs have software instructions performed by one or multiple processors that may be within the same computing device or which may be distributed across multiple computing devices.

The memory 130 may include main memory and/or static memory, where such memories may communicate with each other and the processor 120 via one or more buses. The memory 130 may be implemented by any number, type and combination of random access memory (RAM) and read-only memory (ROM), for example, and may store various types of information, such as software algorithms, artificial intelligence (AI) machine learning algorithms/models, and computer programs, all of which are executable by the processor 120. The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 130 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 130 may store software instructions and/or computer readable code that enable performance of various functions. The memory 130 may be secure and/or encrypted, or unsecure and/or unencrypted. The system 100 may also include database(s) (not shown) for storing information that may be used by the various software modules of the memory 130.

The processor 120 may include or have access to an artificial intelligence (AI) engine, which may be implemented as software that provides artificial intelligence (e.g., reconstruction algorithms) and applies machine learning described herein. The AI engine may reside in any of various components in addition to or other than the processor 120, such as the memory 130, an external server, and/or the cloud, for example. When the AI engine is implemented in a cloud, such as at a data center, for example, the AI engine may be connected to the processor 120 via the internet using one or more wired and/or wireless connection(s).

The user interface 122 provides information and data output by the processor 120 and/or the memory 130 to the user and/or receives information and data input by the user. That is, the user interface 122 enables the user to enter data and to control or manipulate aspects of the processes described herein, and also enables the processor 120 to indicate the effects of the user's control or manipulation. All or a portion of the user interface 122 may be implemented by a graphical user interface (GUI) viewable on the display 124. The user interface 122 may include one or more interface devices, such as a mouse, a keyboard, a trackball, a joystick, a microphone, a video camera, a touchpad, a touchscreen, voice or gesture recognition captured by a microphone or video camera, for example.

The display 124, also referred to as a diagnostic viewer, may be a monitor such as a computer monitor, a television, a liquid crystal display (LCD), an organic light emitting diode (OLED), a flat panel display, a solid-state display, or a cathode ray tube (CRT) display, or an electronic whiteboard, for example. The display 124 includes a screen 126 for viewing internal images of a subject (patient), indicated by the skin 150, along with various features described herein to assist the user in accurately and efficiently reading the medical images, as well as the GUI 128 to enable the user to interact with the displayed images and features. The user is able to personalize the various features of the GUI 128, discussed below, by creating specific alerts and reminders, for example.

Referring to the memory 130, image module 131 is configured to receive and process image data of images received from the imaging system 140, including one or more multi-band reflectance images of the skin 150 from the spatially resolved camera 144. The received images may optionally include RGB-value images and grey-value images from the RGB-value camera 146 and the grey-value camera 148. Alternatively, the image module 131 may be configured to derive RGB-value images and/or grey-value images from the multi-band reflectance images provided by the spatially resolved camera 144. The live images may be received during a contemporaneous imaging session of the subject, or may be stored images retrieved from storage, such as a database or the memory 130, without departing from the scope of the present teachings.

Delineation module 132 is configured to delineate one or more spatial components of a multi-band reflectance image of the skin 150, where the spatial components have substantially homogenous optical properties, respectively. The spatial components refer to regions of interest within the multi-band reflectance image having similar dependence of reflectance and/or absorbance properties on the hemodynamic parameters, respectively. The spatial components are delineated in a signal space of the spatially resolved camera 144, and/or in an RGB-value image and/or grey-value image, discussed above.

The delineated spatial components may be clinically relevant or clinically irrelevant. Clinically relevant spatial components correspond to homogenous regions of interest on the skin 150, which have substantially homogenous optical properties and are the subject of analysis, including healthy tissue, wounds, and surface veins, for example. Clinically irrelevant spatial components are disturbances, which are minor features generally not of interest and visually interrupt the otherwise homogenous regions of interest captured by the clinically relevant spatial components. The clinically irrelevant spatial components may include hairs, moles, pimples and freckles, for example. In an example, the perfusion of the clinically irrelevant spatial components, if at all presence, is not clinically relevant for assessing the perfusion properties of the limb of interest.

The delineation module 132 delineates at least one clinically relevant spatial component of the multi-band reflectance image of the skin 150. The clinically irrelevant spatial component(s) do not need to be delineated, unless it is desired to remove them from the multi-band reflectance image, e.g., by inpainting, so as not to interfere with the analysis of the delineated at least one clinically relevant spatial component, as discussed below. The delineated clinically relevant spatial components may be highlighted (e.g., outlined) and displayed on the display 124 overlaid on the multi-band reflectance image of the skin 150, or on the RGB-value and/or grey-value image.

In various embodiments, the delineation of the at least one clinically relevant spatial component may be performed automatically or semi-automatically using a delineation algorithm provided by the delineation module 132. The delineation algorithm segments optically similar portions of the multi-band reflectance image, such as wound tissue versus normal skin. The segmentation may be performed on the multi-band reflectance image, or alternatively on an RGB-value image and/or a grey-value image spatially aligned with the multi-band reflectance image. The delineation algorithm may include a machine-learning model, such as a convolutional neural network (CNN), for example, for performing semantic segmentation. The machine-learning model may be trained and optimized based on a set of annotated (labeled) data samples. Alternatively, the delineation algorithm may be based on purpose-driven heuristics, such as vesselness filters or region-growing, for example. In one example, a U-Net architecture is trained to semantically segment one or more spatial components by optimizing its learnable parameters to minimize a combination of a cross-entropy and DICE loss on a training set of input image and target binary mask(s) pairs using known stochastic optimization methods. The resulting segmented classes provided by the machine-learning model are known to be either clinically relevant or clinically irrelevant. Accordingly, the same machine-learning model may be used to delineate both clinically relevant and clinically irrelevant spatial components, if desired, as part of the delineation process.

In alternative embodiments, the delineation may be carried out manually by the user via a GUI while viewing the multi-band reflectance image on the display 124, in which case the delineation module 132 receives the delineation information via the user interface 122. The user may outline or otherwise mark the clinically relevant spatial components to be delineated using known techniques. Marking the clinically relevant spatial components may include labeling a type of each of the clinically relevant spatial components. The type may include healthy tissue, wound or surface vein, for example. The user may likewise outline or otherwise mark the clinically irrelevant spatial components, if desired, to be delineated using known techniques.

FIG. 2 shows illustrative images of a human foot subjected to delineation of clinically relevant spatial components, according to a representative embodiment. Referring to FIG. 2, a multi-band reflectance image is obtained of foot 210, which has a gaping wound 215 with exposed wound tissue. The multi-band reflectance image is delineated by the delineation module 132 using a delineation CNN, for example, into healthy skin spatial component 220 corresponding to uninjured areas on the bottom of the foot 210 and wound tissue spatial component 230 corresponding to the wound 215.

The spatial components, delineated by a CNN, may also be used for computing dedicated summary measures for a monitoring use case. In the depicted example, summary measures of oxygenation (O₂) over time (t) are provided by spatially accumulating (e.g. averaging) the reconstructed perfusion parameter over each spatial component, where the mean oxygenation for the healthy skin spatial component 220 is shown by graph 225, and the mean oxygenation for the wound tissue spatial component 230 is shown by graph 235. Of course, summary measures of other skin perfusion parameters, such as blood volume and blow flow, may be incorporated without departing from the scope of the present teachings.

In an alternative embodiment, a preliminary perfusion parameter reconstruction algorithm is performed on a multi-band reflectance image to provide an estimated perfusion parameter map on which the delineation is performed. In one example, a correspondingly trained CNN maps such a preliminary estimate for the blood content map, for example, to a binary mask of surface veins as a spatial component. The preliminary blood-content estimate may be used as an input for a segmentation network, which can be advantageous over using a corresponding RGB-value image since surface veins may appear much more visible in the preliminary blood-content estimate. The resulting binary mask of the surface veins is then used for applying a blood-content reconstruction algorithm tailored to the reflectance characteristics of surface veins only to the surface-vein spatial component. Only the blood-content prediction of this tailored algorithm is used for any subsequent analysis. In a further example, the aforementioned surface-vein segmentation algorithm can be trained and applied on both RGB-value images and preliminary blood-content map estimates, being concatenated along the image channel dimension.

As an example, FIG. 3 shows illustrative images of delineated clinically relevant and clinically irrelevant spatial components of an estimated perfusion parameter map, according to a representative embodiment. Referring to FIG. 3, a preliminary perfusion parameter reconstruction algorithm is applied to the acquired multi-band reflectance image to provide an estimated perfusion parameter map, such as a heatmap of blood content as shown in image 301. The delineation algorithm of the delineation module 132 then delineates the spatial components in the estimated perfusion parameter map, including a clinically irrelevant spatial component (pimple 322) and two clinically relevant spatial components (surface veins 321 and the spatial complement 323 of the segmented surface veins 321 and the segmented pimple 322 which may be regarded as “normal” skin tissue), as shown in image 302. In comparison, the multi-band reflectance image is given by intensities of reflected light in different wavelength bins at each pixel. Therefore, the input of the segmentation algorithm (and corresponding training data) is different for the estimated perfusion parameter map. Inpainting may be performed on the clinically irrelevant spatial component. Image 303 shows the multi-band reflectance image after delineating the clinically relevant and irrelevant spatial components and applying inpainting. As shown in image 303, the pimple 322 has been inpainted, and therefore is no longer visible. Also, image 303 has enhanced color saturation as compared to image 301, indicating refinement of the estimated perfusion parameter map.

Reconstruction module 133 is configured to perform reconstruction on each of the delineated clinically relevant spatial components of the multi-band reflectance image in order to estimate corresponding perfusion parameters of the skin 150. The reconstruction module 133 incorporates tailored reconstruction algorithms, which are specific to the delineated clinically relevant spatial components, respectively. Depending on the application and spatial extent of the regional components, the tailored reconstruction algorithms may be applied pixel-wise, but also surface patch-wise taking the neighborhood spectra into account. Therefore, for each class of the clinically relevant spatial components (e.g. healthy tissue, wounds, surface veins), a corresponding reconstruction algorithm is individually trained for predicting the perfusion parameters of interest. The corresponding reconstruction algorithms may be the same reconstruction algorithm trained differently for the different clinically relevant spatial components, or may be different reconstruction algorithms, e.g. artificial neural networks with different architectures. Also, some clinically relevant spatial components, such as surface veins, for example, may by predicted using a vesselness filter. Each reconstruction algorithm may be realized by a fully-connected neural network operating on the discrete input spectrum of each pixel in a corresponding spatial component of the of the multi-band reflectance image, for example. The reconstruction algorithm may then be evaluated on the spectrum of any pixel, for example.

In an alternative embodiment, the reconstruction module 133 may incorporate a convolutional architecture, i.e. a convolutional neural network (CNN). The underlying convolutions may act on the local frequency spectrum of a pixel of the respective spatial component. In another example, the underlying convolutions may act in the spatial domain, i.e., the convolutional kernels slide over the respective spatial component and operate on the reflectance spectra of a group of pixels in the first layer of the CNN. In this way, a small region of input pixels can contribute to the final perfusion parameter prediction, which may require restricting reconstruction results to pixels of the respective spatial component in the end. Accordingly, smoother perfusion parameter predictions may be realized and input noise in the reflectance spectra may be compensated. In another embodiment, a convolutional architecture operating in the spatial domain may be combined with convolutions sliding along the frequency (channel) dimension or also with fully-connected layers.

Thus, the reconstruction module 133 outputs one or more estimated perfusion parameters of the skin 150 for each of the reconstructed clinically relevant spatial components. The estimated perfusion parameters may be displayed on the display 124 overlaid on the multi-band reflectance image of the skin 150, or overlaid on the RGB-value image and/or the grey-value image of the skin 150, enabling the user to easily associate the estimated perfusion parameters with the clinically relevant spatial components. The estimated perfusion parameters provided by the tailored reconstruction algorithms are more accurate and/or more clinically meaningful as compared to perfusion parameters provided by conventional techniques.

In an embodiment, the estimated perfusion parameters may be mapped back into a perfusion parameter map across the imaged skin 150. That is, when a tailored reconstruction algorithm is chosen depending on the class of clinically relevant spatial component (e.g., treated per pixel of the imaging system 140), as discussed above, the estimation module 134 may map the resulting reconstructed clinically relevant spatial components as regional components back into a perfusion parameter map across the imaged skin 150. The perfusion parameter map also may be displayed on the display 124 overlaid on the multi-band reflectance image of the skin 150, or the RGB-value image and/or the grey-value image of the skin 150 enabling the user to easily associate the estimated perfusion parameters with the clinically relevant spatial parameters.

Also, as discussed above with reference to FIG. 3, regional reconstruction algorithm selection for regional or pixelwise prediction may be performed based on an estimated parameter map estimate that originates from a preliminary perfusion parameter reconstruction algorithm. For example, the estimated parameter map may be for predicting hemoglobin content of the skin 150. Certain clinically relevant spatial components, such as surface veins, for example, may be more prominent in this preliminary hemoglobin estimate so that component segmentation, particularly with purpose-driven heuristics (e.g., a vesselness filter), is more accurate or robust. This approach is effectively a cascade that bases a second reconstruction using the same regionally dependent reconstruction algorithms, as a refinement, on the prediction of the generic reconstruction algorithm, as shown in FIG. 3, for example.

Monitoring module 134 is configured to monitor estimated perfusion parameters provided by the estimation module 134 that are spatially accumulated over time, e.g., for larger clinically relevant spatial components such as healthy skin and wounds. That is, after the reconstruction module 133 applies the tailored reconstruction algorithms for each clinically relevant spatial component, as discussed above, the monitoring module 134 accumulates corresponding perfusion parameter maps, which may be observed as a semantically decomposed perfusion parameter vector over time. For example, a perfusion parameter vector may indicate wound versus healthy skin, as shown in FIG. 2. The perfusion parameter maps may be accumulated over each clinically relevant spatial component by averaging or by percentile analysis, for example, and displayed on the display 124 overlaid on the multi-band reflectance image of the skin 150.

Inpainting module 135 is configured to perform inpainting of clinically irrelevant spatial components, such as hair, moles, pimples and freckles, for example, using known inpainting algorithms. The clinically irrelevant spatial components may be segmented and ignored during selection and application of the tailored reconstruction algorithms to the clinically relevant spatial components. The resulting perfusion parameter maps or spectral input spaces exhibit holes at the locations of the ignored clinically irrelevant spatial components. The inpainting module 135 spatially inpaints the holes on the display 124 to hide (e.g., make transparent) the ignored clinically irrelevant spatial components from the user, showing a smooth surface map as a result. The inpainting generally enables cleaner visualizations of the perfusion parameters

In an embodiment, the inpainting module 135 inpaints the measured reflectance spectrum from neighboring relevant pixels into clinically irrelevant spatial components. This may be done before reconstruction, in which case the reconstruction module 133 performs the reconstruction on an effectively enhanced input, where the clinically irrelevant spatial components have been artificially promoted to being clinically relevant by virtue of the inpainting. In another embodiment, the reconstruction module 133 performs the reconstruction only on the delineated clinically relevant spatial components, and the inpainting module 135 then inpaints the clinically irrelevant spatial components using the reconstructed perfusion parameters of neighboring pixels of a clinically relevant spatial component.

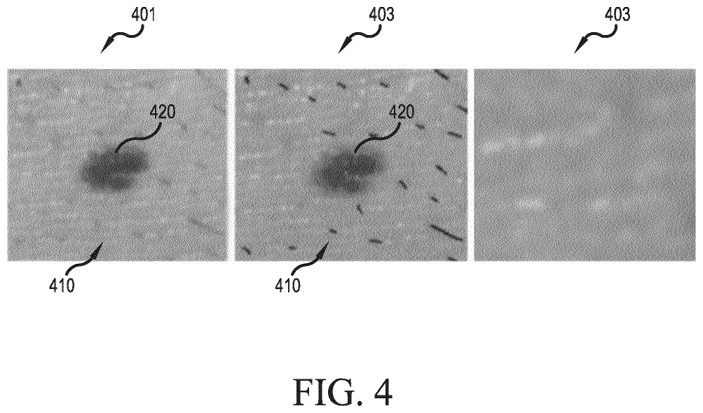

FIG. 4 shows illustrative images of spatially irrelevant spatial components subjected to inpainting, according to a representative embodiment. Referring to FIG. 4, a multi-band reflectance image of a skin with hair 410 and a mole 420 is shown in image 401. Image 402 is a perfusion map estimated from the estimation module 134 using a generic model irrespective of spatial components, where the hair 410 and the mole 420 stand out. Image 403 is the perfusion map after the hair 410 and the mole 420 have been inpainted by the inpainting module 135, where the corresponding clinically irrelevant spatial components have been filled in using information from reconstructed perfusion parameters of neighboring pixels. In the depicted embodiment, the hair 410 and the mole 420 have been delineated by the delineation module 132, and ignored during reconstruction by the reconstruction module 133.

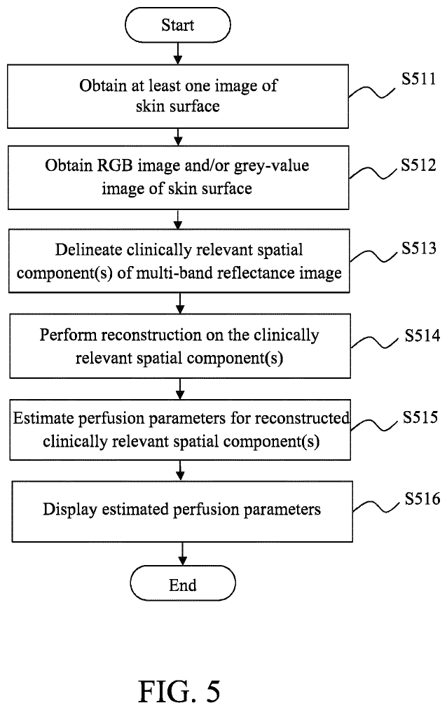

FIG. 5 is a flow diagram of a method for analyzing perfusion parameters of skin, according to a representative embodiment. The method may be implemented by the system 100, discussed above, under control of the processor 120 executing instructions stored as the various software modules in the memory 130, for example.

Referring to FIG. 5, at least one image of the skin of a subject is obtained in block S511. The image may be received directly from a spatially resolved camera (e.g., spatially resolved camera 144) of a medical imaging device/modality, or may be retrieved from a database, for example, in which the image had been previously stored. The at least one image includes a multi-band reflectance image, and may further include an RGB-value image and/or a grey-value image spatially aligned with the multi-band reflectance image. The RGB-value image and/or the grey-value image of the skin may be acquired separately or derived from the multi-band reflectance image. The multi-band reflectance image includes at least two wave-lengths in an optical, near infrared and/or near ultra-violet range, and may be a multi-spectral image or a hyperspectral image, for example.

In block S512, optionally, an RGB-value image and/or a grey-value image of the skin is obtained. The RGB-value image and/or the grey-value image are spatially aligned with the multi-band reflectance image. So, the RGB-value image and/or the grey-value image may be derived from the multi-band reflectance image, or may be acquired separately from the multi-band reflectance image using an RGB-value camera (e.g., RGB-value camera 146) and/or a grey-value camera (e.g., grey-value camera 148), respectively, in addition to or as part of the spatially resolved camera. When a separate RGB-value camera or grey-value camera is used, the respective images may be spatially aligned with the multi-band reflectance image using a known, fixed displacement and viewing directions of the RGB-value camera or the grey-value camera and the spatially resolved camera for multi-band reflectance imaging, plus measuring distances to corresponding surface points and triangulation. Alternatively, the images from the RGB-value camera or the grey-value camera may be spatially aligned with the multi-band reflectance image through calibration.

In block S513, at least one clinically relevant spatial component of the multi-band reflectance image of the skin is delineated. The at least one clinically relevant spatial component corresponds to a homogenous region of interest on the skin having substantially homogenous optical properties, as indicated by the multi-band reflectance image. The at least one clinically relevant spatial component are the subject of analysis, and may include healthy tissue, wounds, and surface veins, for example.

In an embodiment, delineating the at least one clinically relevant spatial component may include performing automatic segmentation on a local backscatter spectrum of the multi-band reflectance image. In the event an RGB-value image and/or a grey-value image has been obtained, delineating the at least one clinically relevant spatial component may include performing automatic segmentation on the RGB-value image and/or the grey-value image, respectively. In these cases, known automatic segmentation algorithms may be incorporated. For example, the automatic segmentation may be performed by applying a CNN that is optimized based on a set of annotated data samples from the at least one clinically relevant spatial component. Alternatively, the automatic segmentation may be performed by applying purpose-driven heuristics to the at least one clinically relevant spatial component.

In an embodiment, delineating the at least one clinically relevant spatial component may include manually identification by the user the at least one clinically relevant spatial component, e.g., using a GUI. The manual identification may include annotating (labeling) a type of the at least one clinically relevant spatial component. Types of clinically relevant spatial components may include healthy tissue, wound, surface vein, and the like.

In an embodiment, perfusion parameters may be initially estimated by applying an initial perfusion parameter reconstruction algorithm to the multi-band reflectance image. The initial perfusion parameter reconstruction algorithm may be the same as a tailored reconstruction algorithm (e.g., CNN) used for performing reconstruction, discussed below with reference to block S514. However, the initial perfusion parameter reconstruction algorithm would be trained on a broader training set (e.g., not specialized to surface veins) or a narrower training set (e.g., no surface veins included). Segmentation of the estimated perfusion parameter map is then performed to identify the at least one clinically relevant spatial component for delineation.

In block S514, reconstruction is performed on the at least one clinically relevant spatial component using a corresponding at least one tailored reconstruction algorithm, respectively. The at least one tailored reconstruction algorithm is specific to the at least one clinically relevant spatial component. As discussed above, the tailored reconstruction algorithms for different clinically relevant spatial components may be the same reconstruction algorithm but trained differently. Or, the tailored reconstruction algorithms for different clinically relevant spatial components may be different reconstruction algorithms, e.g. CNNs of different architecture.

In block S515, perfusion parameters of the skin are estimated for the at least one reconstructed clinically relevant spatial component. The estimated perfusion parameters for each of the at least one reconstructed clinically relevant spatial component may include any of one or more perfusion parameters, including blood volume, blood flow and blood oxygenation, depending on clinical requirements. The perfusion parameters may be estimated by translating the reflectance spectra of the pixel(s) of the at least one clinically relevant spatial component into a corresponding value of the subject perfusion parameter. In an embodiment, the estimated perfusion parameters may be mapped back into a perfusion parameter map across the imaged skin.

In block S516, the estimated perfusion parameters for the at least one reconstructed clinically relevant spatial component are displayed. The estimated perfusion parameters may be displayed in a spatially resolved manner as an overlay on the multi-band reflectance image, or as an overlay on the RGB-value image and/or the grey-value image. Also, the estimated perfusion parameters may be displayed in the form of a perfusion parameter map. In an embodiment, the delineated and/or reconstructed at least one clinically relevant spatial component may be displayed along with the estimated perfusion parameters.

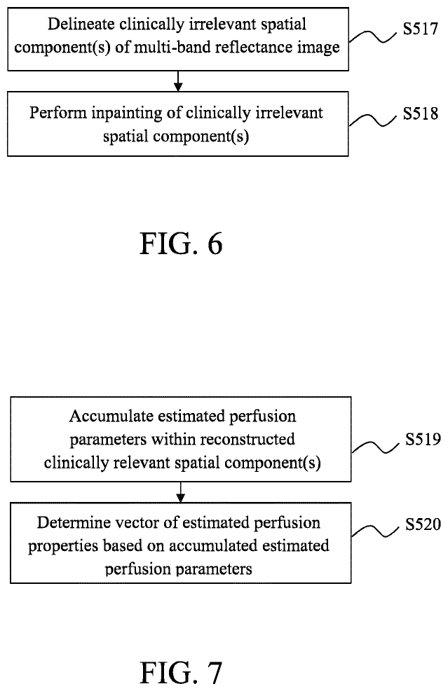

FIG. 6 is a flow diagram of a method of inpainting the at least one clinically relevant spatial component for analyzing perfusion parameters of skin, according to a representative embodiment. The method likewise may be implemented by the system 100, discussed above, under control of the processor 120 executing instructions stored as the various software modules in the memory 130, for example. The method of FIG. 6 may be performed in addition to the steps described above with reference to FIG. 5.

Referring to FIG. 6, at least one clinically irrelevant spatial component of the multi-band reflectance image of the skin is delineated in block S517. The at least one clinically irrelevant spatial component is a disturbance that is generally not of interest, and visually interrupts the otherwise homogenous region of interest captured by the at least one clinically relevant spatial component. The clinically irrelevant spatial components may include hairs, moles, pimples and freckles, for example.

As discussed above, delineating the at least one clinically irrelevant spatial component may include automatic or semi-automatic segmentation using a known delineation algorithm. The segmentation may be performed on the multi-band reflectance image, or alternatively based on the RGB-value image and/or the grey-value image spatially aligned with the multi-band reflectance image. The delineation algorithm may include a machine-learning model, such as a CNN, for example, for performing semantic segmentation.

Alternatively, delineating the at least one clinically irrelevant spatial component may be performed done manually by the user via a GUI using the multi-band reflectance image, or alternatively using the RGB-value image and/or the grey-value image, where the manual delineation is received via the GUI for further processing. The user may outline or otherwise mark the clinically relevant spatial components to be delineated using known techniques.

In block S518, inpainting the at least one clinically irrelevant spatial component is performed. This may include inpainting the measured reflectance spectrum from neighboring relevant pixels into the at least one clinically irrelevant spatial component, which may be done before reconstructing the at least one clinically relevant spatial component in block S514. In this case, the inpainted at least one clinically irrelevant spatial component is reconstructed along with the at least one clinically relevant spatial component. Alternatively, inpainting the at least one clinically irrelevant spatial component may include inpainting after reconstructing the at least one clinically relevant spatial component in block S514. In this case, only the at least one delineated clinically relevant spatial component is reconstructed in block S514, leaving the at least one clinically irrelevant spatial component untouched. The inpainting is then performed on the at least one clinically irrelevant spatial component using reconstructed perfusion parameters of neighboring pixels.

FIG. 7 is a flow diagram of a method of monitoring estimated perfusion parameters that are spatially accumulated over time for analyzing perfusion parameters of skin, according to a representative embodiment. The method likewise may be implemented by the system 100, discussed above, under control of the processor 120 executing instructions stored as the various software modules in the memory 130, for example. The method of FIG. 7 may be performed in addition to the steps described above with reference to FIG. 5.

Referring to FIG. 7, in block S519, estimated perfusion parameters within the at least one reconstructed clinically relevant spatial component are spatially accumulated over time for each frame. That is, after the delineated at least one clinically relevant spatial component is reconstructed in block S514, corresponding perfusion parameter maps showing the estimated perfusion parameters are accumulated. In block S520, a vector of the estimated perfusion properties is determined based on the accumulated estimated perfusion parameters. That is, the accumulated perfusion parameter maps may be observed as a semantically decomposed perfusion parameter vector over time.

In accordance with various embodiments of the present disclosure, the methods described herein may be implemented using a hardware computer system that executes software programs stored on non-transitory storage mediums. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although analyzing perfusion parameters of skin has been described with reference to exemplary embodiments, it is understood that the words that have been used are words of description and illustration, rather than words of limitation. Changes may be made within the purview of the appended claims, as presently stated and as amended, without departing from the scope and spirit of interventional procedure optimization in its aspects. Although analyzing perfusion parameters of skin has been described with reference to particular means, materials and embodiments, analyzing perfusion parameters of skin is not intended to be limited to the particulars disclosed; rather analyzing perfusion parameters of skin extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method for analyzing perfusion parameters of skin, the method comprising:
acquiring a multi-band reflectance image of skin of a subject;
delineating at least one clinically relevant spatial component of the multi-band reflectance image of the skin, wherein the at least one clinically relevant spatial component has substantially homogenous optical properties;
performing reconstruction on the at least one clinically relevant spatial component using a corresponding at least one tailored reconstruction algorithm, respectively, wherein the at least one tailored reconstruction algorithm is specific to the at least one clinically relevant spatial component; and
outputting estimated perfusion parameters of the skin for the at least one reconstructed clinically relevant spatial component from the at least one tailored reconstruction algorithm.

2. The method of claim 1, further comprising:
displaying the estimated perfusion parameters for the at least one reconstructed clinically relevant spatial component in a spatially resolved manner as an overlay on the multi-band reflectance image.

3. The method of claim 1, further comprising:
mapping the estimated perfusion parameters back into a perfusion parameter map across the skin.

4. The method of claim 1, further comprising:
receiving an RGB-value image and/or a grey-value image of the skin,
wherein the RGB-value image and/or the grey-value image are spatially aligned with the multi-band reflectance image; and
displaying the estimated perfusion parameters for the at least one reconstructed clinically relevant spatial component in a spatially resolved manner as an overlay on the RGB-value image and/or the grey-value image.

5. The method of claim 1, further comprising:
accumulating the estimated perfusion parameters within the at least one reconstructed clinically relevant spatial component; and
determining a vector of the estimated perfusion properties of the at least one clinically relevant spatial component based on the accumulated estimated perfusion parameters.

6. The method of claim 5, wherein accumulating the estimated perfusion parameters within the at least one reconstructed clinically relevant spatial component comprises taking an average, a mean, a median, a quantile and/or a variance of the estimated perfusion parameters within the at least one reconstructed clinically relevant spatial component.

7. The method of claim 5, wherein the at least one clinically relevant spatial component of the multi-band reflectance image of the skin is delineated automatically.

8. The method of claim 1, wherein delineating the at least one clinically relevant spatial component comprises performing automatic segmentation on a local backscatter spectrum of the multi-band reflectance image.

9. The method of claim 4, wherein delineating the at least one clinically relevant spatial component comprises performing automatic segmentation on the RGB-value image and/or the grey-value image.

10. The method of claim 1, wherein delineating the at least one clinically relevant spatial component comprises performing automatic segmentation by applying a convolutional neural network that is optimized based on a set of annotated data samples from the at least one clinically relevant spatial component, or by applying purpose-driven heuristics to the at least one clinically relevant spatial component.

11. The method of claim 1, further comprising:
initially estimating perfusion parameters by applying an initial perfusion parameter reconstruction algorithm to the multi-band reflectance image to provide an estimated perfusion parameter map; and
performing segmentation of the estimated perfusion parameter map to identify the at least one clinically relevant spatial component for delineation.

12. The method of claim 1, further comprising:
delineating at least one clinically irrelevant spatial component of the multi-band reflectance image of the skin, wherein the at least one clinically irrelevant spatial component comprises a disturbance; and
inpainting the at least one clinically irrelevant spatial component.

13. The method of claim 1, wherein each of the tailored reconstruction algorithms is realized by a neural network operating on a discrete input spectrum of the at least one pixel of the multi-band reflectance image.

14. A system for analyzing perfusion parameters of skin, the system comprising:
an imaging system configured to acquire a multi-band reflectance image of skin;
at least one processor coupled to the imaging system to receive the multi-band reflectance image of the skin of a subject; and
at least one non-transitory memory storing instructions that, when executed by the at least one processor, cause the at least one processor to:
delineate at least one clinically relevant spatial component of the multi-band reflectance image of the skin, wherein the at least one clinically relevant spatial component has substantially homogenous optical properties;
perform reconstruction on the at least one clinically relevant spatial component using a corresponding at least one tailored reconstruction algorithm, respectively, wherein the at least one tailored reconstruction algorithm is specific to the at least one clinically relevant spatial component;
output estimated perfusion parameters of the skin for the at least one reconstructed clinically relevant spatial component from the at least one tailored reconstruction algorithm; and
a display configured to display the estimated perfusion parameters for the at least one reconstructed clinically relevant spatial component in a spatially resolved manner.

15. The system of claim 14, further comprising:
an RGB-value camera and/or a grey-value camera configured to provide an RGB-value image and/or a grey-value image of the skin, respectively, wherein the RGB-value image and/or the grey-value image are spatially aligned with the multi-band reflectance image, and
wherein the display is further configured to display the estimated perfusion parameters for the at least one reconstructed clinically relevant spatial component in a spatially resolved manner.

16. The system of claim 15, wherein the display is configured to display the estimated perfusion parameters for the at least one reconstructed clinically relevant spatial component as an overlay on the multi-band reflectance image, or as an overlay on the RGB-value image and/or the grey-value image.

17. The system of claim 14, wherein the instructions further cause the at least one processor to:
accumulate the estimated perfusion parameters within the at least one reconstructed clinically relevant spatial component; and
determine a vector of the estimated perfusion properties of the at least one clinically relevant spatial component based on the accumulated estimated perfusion parameters.

18. The system of claim 14, wherein the instructions further cause the at least one processor to:
initially estimate perfusion parameters by applying an initial perfusion parameter reconstruction algorithm to the multi-band reflectance image to provide an estimated perfusion parameter map; and
perform segmentation of the estimated perfusion parameter map to identify the at least one clinically relevant spatial component for delineation.

19. The system of claim 14, wherein the instructions further cause the at least one processor to:
delineate at least one clinically irrelevant spatial component of the multi-band reflectance image of the skin, wherein the at least one clinically irrelevant spatial component comprises a disturbance; and
inpaint the at least one clinically irrelevant spatial component.

20. A non-transitory computer readable medium that stores instructions for analyzing perfusion parameters of skin of a subject that, when executed by at least one processor, cause the at least one processor to:
receive a multi-band reflectance image of the skin;
delineate at least one clinically relevant spatial component of the multi-band reflectance image of the skin, wherein the at least one clinically relevant spatial component has substantially homogenous optical properties;
perform reconstruction on the at least one clinically relevant spatial component using a corresponding at least one tailored reconstruction algorithm, respectively, wherein the at least one tailored reconstruction algorithm is specific to the at least one clinically relevant spatial component;
output estimated perfusion parameters of the skin for the at least one reconstructed clinically relevant spatial component from the at least one tailored reconstruction algorithm; and
cause a display of the estimated perfusion parameters for the at least one reconstructed clinically relevant spatial component in a spatially resolved manner.
